(19) United States
(12) Patent Application Publication
(10) Pub. No.: US 2021/0277005 A1
(43) Pub. Date: Sep. 9, 2021

(54) PROANGIOGENIC COMPOSITIONS AND METHODS OF USE

(71) Applicant: University of North Dakota, Grand Forks, ND (US)

(72) Inventor: Mikhail Y. Golovko, Grand Forks, ND (US)

(21) Appl. No.: 17/190,177

(22) Filed: Mar. 2, 2021

Related U.S. Application Data
(60) Provisional application No. 62/986,262, filed on Mar. 6, 2020.

(51) Int. Cl. C07D 473/34; A61P 25/00

(52) U.S. Cl. CPC C07D 473/34; A61P 25/00

(57) ABSTRACT

Various embodiments disclosed relate to a pharmaceutical composition. The composition includes the structure according to Formula I or pharmaceutically acceptable ester thereof, prodrug thereof, or a pharmaceutically acceptable salt thereof:

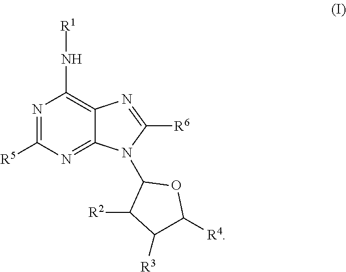

In Formula I, R¹, R², R³, R⁴, R⁵, and R⁶, are independently selected from the group consisting of —H, —OH, —COOH, and substituted or unsubstituted (C₁-C₂₀)hydrocarbyl.

### CROSS-REFERENCE TO RELATED APPLICATIONS

This application claims the benefit of priority to U.S. Provisional Patent Application Ser. No. 62/986,262 entitled “PROANGIOGENIC COMPOSITIONS AND METHODS OF USE,” filed Mar. 6, 2020, the disclosure of which is incorporated herein in its entirety by reference.

### BACKGROUND

Angiogenesis is the physiological process through which new blood vessels form from pre-existing vessels, formed in the earlier stage of vasculogenesis. Angiogenesis continues the growth of the vasculature by processes of sprouting and splitting existing blood vessel networks.

### SUMMARY OF THE DISCLOSURE

Various embodiments disclosed relate to a pharmaceutical composition. The composition includes the structure according to Formula I or pharmaceutically acceptable ester thereof, prodrug thereof, or a pharmaceutically acceptable salt thereof:

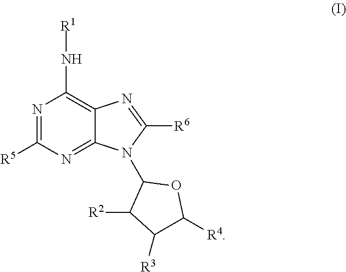

In Formula I R¹, R², R³, R⁴, R⁵, and R⁶, are independently selected from the group consisting of —H, —OH, —COOH and substituted or unsubstituted (C₁-C₂₀)hydrocarbyl.

Various embodiments disclosed relate to a pharmaceutical composition for promoting angiogenesis. The composition includes the structure according to Formula I or pharmaceutically acceptable ester thereof, prodrug thereof, or a pharmaceutically acceptable salt thereof:

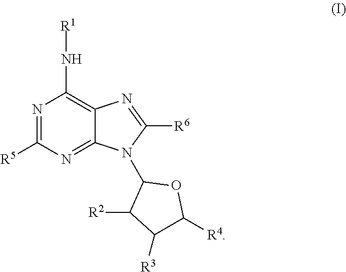

In Formula I R¹, R², R³, R⁴, R⁵, and R⁶, are independently selected from the group consisting of —H, —OH, —COOH and substituted or unsubstituted (C₁-C₂₀)hydrocarbyl.

Various embodiments disclosed relate to a method for promoting angiogenesis. The method includes administering a composition. The composition includes the structure according to Formula I or pharmaceutically acceptable ester thereof, prodrug thereof, or a pharmaceutically acceptable salt thereof:

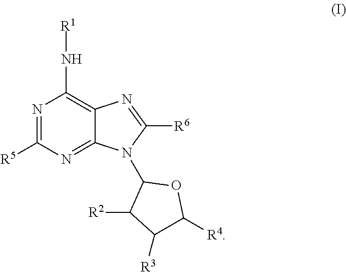

In Formula I R¹, R², R³, R⁴, R⁵, and R⁶, are independently selected from the group consisting of —H, —OH, —COOH, and substituted or unsubstituted (C₁-C₂M)hydrocarbyl.

### BRIEF DESCRIPTION OF THE FIGURES

The drawings illustrate generally, by way of example, but not by way of limitation, various embodiments discussed in the present document.

FIGS. 1A-1D are a series of graph and plots showing the effects of AdSucc treatment to stimulate angiogenesis for brain injury.

FIGS. 2A-2E are a series of graphs showing that AdSucc increases under low energy conditions that require angiogenesis for adaptation.

FIGS. 3A-3F area a series of graphs and plots showing AdSucc Ca²⁺ signaling properties.

FIGS. 4A-4D area a series of graphs and plots showing AdSucc angiogenic properties.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety, for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like, and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃, wherein each R is independently selected, and protonated forms of each, except for —NR₃, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

Brain angiogenesis, or vascular formation from pre-existing vessels, is required for normal organ formation during development, adaptation to low energy conditions e.g. hypoxia and ischemia, and recovery after organ injury such as stroke, brain trauma, or heart attack. Decreased brain angiogenesis may be associated with brain ischemia, neurodegeneration, and damage during aging while increased angiogenesis is linked to tumorigenesis, hypertension, obesity, atherosclerosis, and blindness among other pathologies. Angiogenesis is the process of expanding existing blood vessel networks mainly by sprouting new branches that connect and subsequently remodel into a functional vascular circuit. Brain angiogenesis is critical for tissue growth during development or tumor formation, during inflammation, healing after brain injury including stroke, brain trauma, or heart attack, adaptation to low energy conditions e.g. hypoxia and ischemia, or upon increased physiological metabolic brain demand. Once the new vessels establish nutrient and oxygen supplies that meet the metabolic tissue demand, the vessels will become quiescent. The balance between pro-angiogenic and anti-angiogenic factors tightly regulates angiogenesis in response to metabolic tissue demands. Disturbance of this balance leads to a growing list of diseases. Over proliferation of blood vessels is associated with hypertension, cancers, psoriasis, arthritis, diabetes, obesity, asthma, and atherosclerosis, while defect in angiogenesis can cause heart and brain ischemia, neurodegeneration, hypertension, osteoporosis, respiratory distress, preeclampsia, endometriosis, postpartum cardiomyopathy, and ovarian hyperstimulation syndrome. Therefore, it is suspected that in certain examples, promoting angiogenesis can be helpful in treating some of the aforementioned conditions. Although angiogenesis is described in detail with specific reference to the brain, it is understood that the compounds described herein can be used to promote angiogenesis in other organs. For example, angiogenesis can be promoted in muscles, heart, liver, adipose tissue, lungs, gastrointestinal system, reproductive system, renal system.

In various examples compounds useful in promoting angiogenesis can be used to treat stroke, for example, by promoting blood vessel growth in the brain to aid in recovery. In various example, compounds useful in promoting angiogenesis can be used to treat neurodegeneration in instances where impaired vasculature is a factor (e.g., aging or Alzheimer's disease). In various examples, compounds useful in promoting angiogenesis can be used to treat metabolic brain disorders. In some examples, compounds useful in promoting angiogenesis can be used to treat muscular dystrophy. In some examples, compounds useful in the instant disclosure can be used in treating cancer by reducing angiogenesis in a tumor.

In accordance with various examples of the present disclosure compounds useful in promoting angiogenesis, their tautomers, stereoisomers, or pharmaceutically acceptable salts thereof or esters having a solubility enhancing moieties or prodrugs thereof are provided of the Formula (I):

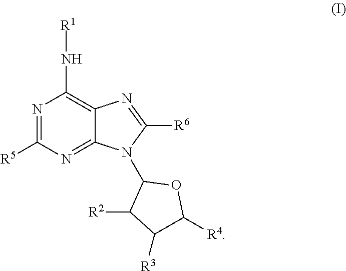

In Formula I, R¹, R², R³, R⁴, R⁵, and R⁶, are independently selected from the group consisting of —H, —OH, —COOH, and substituted or unsubstituted (C₁-C₂₀)hydrocarbyl. In Formula I, R¹, R², R³, R⁴, R⁵, and R⁶, can be independently selected from the group consisting of —H, —OH, substituted or unsubstituted (C₁-C₂M)alkyl, (C₂-C₂D)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

In accordance with various examples of the present disclosure, compounds, their tautomers, stereoisomers, or pharmaceutically acceptable salts thereof or esters having a solubility enhancing moieties or prodrugs thereof are provided of the Formulas (II) and (III), or mixtures thereof:

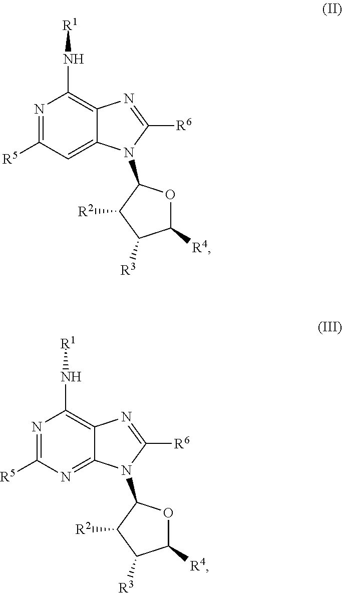

In Formulas II or Ill, R¹, R², R³, R⁴, R⁵, and R⁶, are independently selected from the group consisting of —H, —OH, —COOH, and substituted or unsubstituted (C₁-C₂₀)hydrocarbyl. In Formulas II or III, R¹, R², R³, R⁴, R⁵, and R⁵, can be independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

In accordance with various examples of the present disclosure compounds, their tautomers, stereoisomers, or pharmaceutically acceptable salts thereof or esters having a solubility enhancing moieties or prodrugs thereof are provided of the Formulas (IV) and (V), or mixtures thereof:

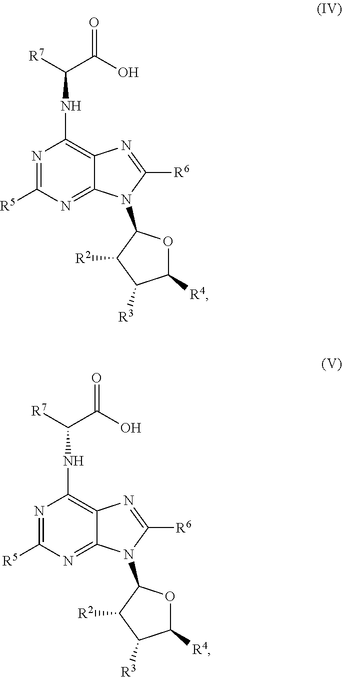

In Formulas IV or V, R², R³, R⁴, R⁵, R⁶, and R⁷, are independently selected from the group consisting of —H, —OH, —COOH, and substituted or unsubstituted (C₁-C₂₀)hydrocarbyl. Moreover, in further examples, in Formulas IV or V, R², R³, R⁴, R⁵, R⁶, and R⁷, are independently selected from the group consisting of —H, —OH, substituted or unsubstituted (C₂-C₂₀)alkyl, (C₂-C₂₀)alkenyl, (C₁-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

In accordance with various examples of the present disclosure compounds, their tautomers, stereoisomers, or pharmaceutically acceptable salts thereof or esters having a solubility enhancing moieties or prodrugs thereof are provided of the Formulas (VI) and (VII), or mixtures thereof:

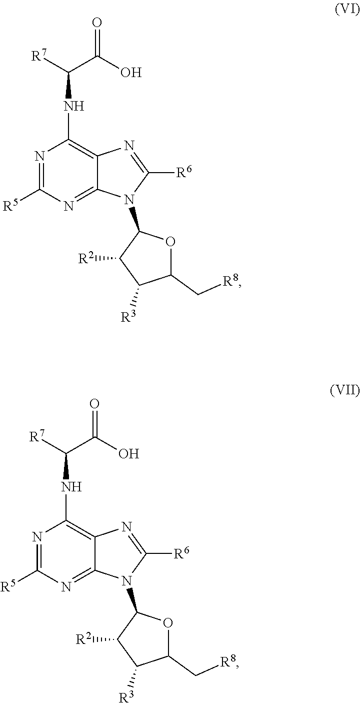

In Formulas VI or VII, R², R³, R⁵, R⁶, R⁷, and R⁸, can be independently selected from the group consisting of —H, —OH, —COOH, and substituted or unsubstituted (C₁-C₂₀)hydrocarbyl. In Formulas VI or VII, R², R³, R⁵, R⁶, R⁷, and R⁸, are independently selected from the group consisting of —H, —OH, substituted or unsubstituted (—C₂₀)alkyl, (C₂-C₂₀)alkenyl, (C₂-C₂₀)alkynyl, (C₁-C₂₀)acyl, (C₁-C₂M)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

In accordance with various examples of the present disclosure compounds, their tautomers, stereoisomers, or pharmaceutically acceptable salts thereof or esters having a solubility enhancing moieties or prodrugs thereof are provided of the Formulas VIII, IX, X, XI, or a mixture thereof, or mixtures thereof:

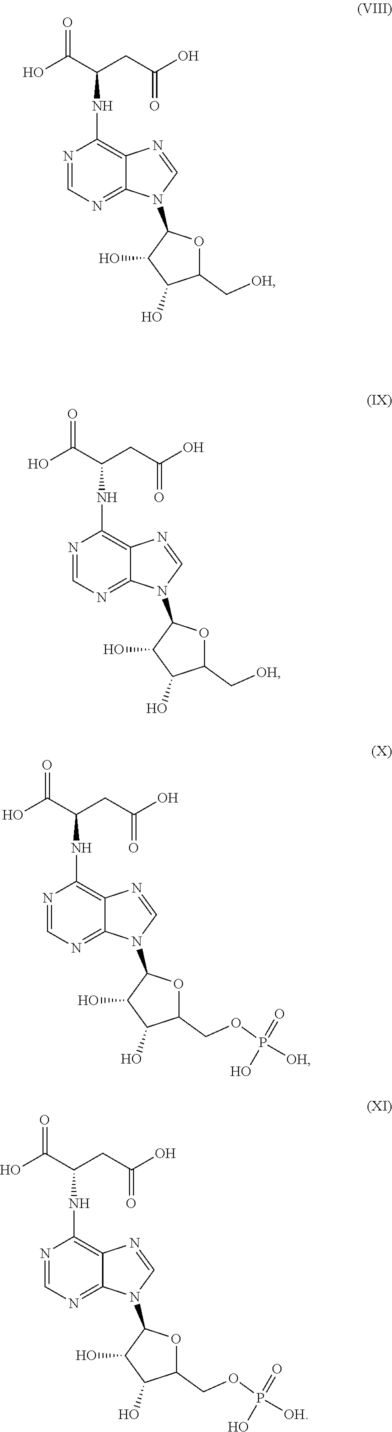 [SMILES: O=C(O)C[C@@H](Nc1ncnc2c1ncn2[C@H]1OC(CO)[C@H](O)[C@@H]1O)C(=O)O ; O=C(O)C[C@H](Nc1ncnc2c1ncn2[C@H]1OC(CO)[C@H](O)[C@@H]1O)C(=O)O ; O=C(O)C[C@@H](Nc1ncnc2c1ncn2[C@H]1OC(COP(=O)(O)O)[C@H](O)[C@@H]1O)C(=O)O ; O=C(O)C[C@H](Nc1ncnc2c1ncn2[C@H]1OC(COP(=O)(O)O)[C@H](O)[C@@H]1O)C(=O)O]

According to various examples, the composition can include adenylsuccinic acid (AdSucc). Adenylsuccinic acid can also be referred to as Adenylosuccinate, 6-Succino-5′-adenylate, 6-Succino-5′-adenylic acid, Adenylsuccinate, Succinyl AMP, Succinyladenosine 5′-monophosphate, Succinyladenosine monophosphorate, Succinyladenosine monophosphoric acid, (2S)-2-({9-[(2R,3R,4S,5R)-3,4-dihydroxy-5-[(phosphonooxy)methyl]oxolan-2-yl]-9H-purin-6-yl}amino)butanedioic acid, (S)-2-((9-((2R,3R,4S,5R)-3,4-Dihydroxy-5-((phosphonooxy)methyl)tetrahydrofuran-2-yl)-9H-purin-6-yl)amino)succinic acid, 2-[9-(3,4-Dihydroxy-5-Phosphonooxymethyl-Tetrahydro-Furan-2-yl)-9h-Purin-6-Ylamino]-Succinic Acid, 2SA, Aspartyl adenylate, L-Aspartic acid, or N-[9-(5-O-phosphono-β-D-ribofuranosyl)-9H-purin-6-yl]aspartic acid.

According to various examples, the composition can include succinyladenosine (SuccAd). Succinyl adenosine can also be referred to as (2S)-2-({9-[(2R,3R,4S,5R)-3,4-Dihydroxy-5-(hydroxymethyl)tetrahydro-2-furanyl]-9H-purin-6-yl}amino)bernsteinsaure, (2S)-2-((9-[(2R,3R,4S,5R)-3,4-Dihydroxy-5-(hydroxymethyl)tetrahydro-2-furanyl]-9H-purin-6-yl)amino)succinic acid, Acid (2S)-2-({9-[(2R,3R,4S,5R)-3,4-dihydroxy-5-(hydroxymethyl)tétrahydro-2-furanyl]-9H-purin-6-yl}amino)succinique, L-Aspartic acid, N-(9-b-D-ribofuranosyl-9H-purin-6-yl)-L-Aspartic acid, (S)—N-(1,2-dicarboxyethyl)-Adenosine, 6-(1,2-Dicarboxyethylamino)-9-b-D-ribofuranosylpurine, 6-(1,2-Dicarboxyethylamino)-9-β-6-ribofuranosylpurine, N-(9-b-D-ribofuranosyl-9H-purin-6-yl)-L-Aspartate, N-(9-β-6-ribofuranosyl-9H-purin-6-yl)-L-Aspartate, N-(9-β-6-ribofuranosyl-9H-purin-6-yl)-L-Aspartic acid, N-9-ribofuranosyl-9H-purin-6-yl-Aspartate, N-9-ribofuranosyl-9H-purin-6-yl-Aspartic acid, (2S)-2-({9-[(2R,3R,4S,5R)-3,4-dihydroxy-5-(hydroxymethyl)oxolan-2-yl]-9H-purin-6-yl)amino)butanedioic acid, (2S)-2-((9-[(2R,3R,4S,5R)-3,4-dihydroxy-5-(hydroxymethyl)oxolan-2-yl]purin-6-yl}amino)butanedioic acid, (2S)-2-((9-[(2R,3R,4S,5R)-3,4-dihydroxy-5-(hydroxymethyl)tetrahydrofuran-2-yl]-9H-purin-6-yl amino)butanedioic acid, 6-(1,2-Dicarboxyethylamino)-9-β-D-ribofuranosylpurine, N-(9-β-D-ribofuranosyl-9H-purin-6-yl)-L-aspartic acid, or N6-Succinyl adenosine

As used herein, the term “pharmaceutically acceptable salts” refers to the nontoxic acid or alkaline earth metal salts of the compounds of Formulas I, II, III, IV, V, VI, VII, VIII, IX, X, or XI. These salts can be prepared in situ during the final isolation and purification of the compounds of Formulas I, II, II, IV, V, VI, VII, VIII, IX, X, or XI, or by separately reacting the base or acid functions with a suitable organic or inorganic acid or base, respectively. Representative salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, pamoate, pectinate, persulfate, 3-phenylproionate, picrate, pivalate, propionate, succinate, sulfate, tartrate, thiocyanate, p-toluenesulfonate and undecanoate. Also, the basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained. Examples of acids which may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the compounds of Formulas I, II, III, IV, V, VI, VII, VIII, IX, X, or XI, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

As used herein, the term “pharmaceutically acceptable ester” refers to esters, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term “pharmaceutically acceptable prodrugs” as used herein refers to those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the disclosure. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

It will be apparent to those skilled in the art that the compounds of the disclosure, including the compounds of Formulas I, III, II, IV, V, VI, VII, VIII, IX, X, or XI or their tautomers, prodrugs and stereoisomers, as well as the pharmaceutically acceptable salts, esters and prodrugs of any of them, may be processed in vivo through metabolism in a human or animal body or cell to produce metabolites. The term “metabolite” as used herein refers to the formula of any derivative produced in a subject after administration of a parent compound. The derivatives may be produced from the parent compound by various biochemical transformations in the subject such as, for example, oxidation, reduction, hydrolysis, or conjugation and include, for example, oxides and demethylated derivatives. The metabolites of a compound of the disclosure may be identified using routine techniques known in the art. See, e.g., Bertolini, G. et al., J. Med. Chem. 40:2011-2016 (1997); Shan, D. et al., J. Pharm. Sci.86(7):765-767; Bagshawe K., Drug Dev. Res. 34:220-230 (1995): Bodor, N., Advances in Drug Res. 13:224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991). It should be understood that individual chemical compounds that are metabolites of the compounds of Formulas (I), (II) or (III) or their tautomers, prodrugs and stereoisomers, as well as the pharmaceutically acceptable salts, esters and prodrugs of any of them, are included within the disclosure.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process. As used herein, the term “treat”, “treating” or “treatment” of any disease or disorder, refers (i) to ameliorating the disease or disorder (i.e., slowing or arresting or reducing the development of the disease or at least one of the clinical symptoms thereof; (ii) to alleviating or ameliorating at least one physical parameter including those which may not be discernible by the patient; (iii) to modulating the disease or disorder, either physically, (e.g., stabilization of a discernible symptom), physiologically, (e.g., stabilization of a physical parameter), or both; or (iv) to preventing or delaying the onset or development or progression of the disease or disorder. In general, the term “treating” or “treatment” describes the management and care of a patient for the purpose of combating the disease, condition, or disorder and includes the administration of a PKC inhibitor to prevent the onset of the symptoms or complications, alleviating the symptoms or complications, or eliminating the disease, condition or disorder.

The compounds of the disclosure are useful in vitro or in vivo in promoting angiogenesis. The compounds may be used alone or in compositions together with a pharmaceutically acceptable carrier or excipient. Suitable pharmaceutically acceptable carriers or excipients include, for example, processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), incorporated herein by reference.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

For purposes of the present disclosure, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose.

The compounds of the present disclosure may be administered orally, parenterally, sublingually, by aerosolization or inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intratarsal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present disclosure can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present disclosure, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in CellBiology, Volume XIV, Academic Press, New York, N.W., p. 33 et seq. (1976).

While the compounds of the disclosure can be administered as the sole active pharmaceutical agent, they can also be used in combination with one or more other agents used in promoting angiogenesis.

The compounds described herein a different than angiogenesis inhibitors. Angiogenesis inhibitors refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS 89:7384 (1992); JNCI 69:475 (1982); Arch. Ophthalmol. 108:573 (1990); Anat. Rec., (238):68 (1994); FEBS Letters 372:83 (1995); Clin, Orthop. 313:76 (1995); J. Mol. Endocrinol. 16:107 (1996); Jpn. J. Pharmacol. 75:105 (1997); Cancer Res. 57:1625 (1997); Cell 93:705 (1998); Intl. J. Mol. Med 2:715 (1998); J. Biol. Chem. 274:9116 (1999)), steroidal anti-inflammatories (such as corticosteroids, mineralocorticoids, dexamethasone, prednisone, prednisolone, methylpred, betamethasone), carboxyamidotriazole, combretastatin A4, squalamine, 6-O-chloroacetyl-carbonyl)-fumagillol, thalidomide, angiostatin, troponin-1, angiotensin II antagonists (see Fernandez et al., J. Lab. Clini. Med 105:141-145 (1985)), and antibodies to VEGF (see, Nature Biotechnology, 17:963-968 (October 1999); Kim et al., Nature, 362:841-844 (1993); WO 00/44777; and WO 00/61186). Other therapeutic agents that modulate or inhibit angiogenesis include agents that modulate or inhibit the coagulation and fibrinolysis systems (see review in Clin. Chem. La. Med 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002).

### Examples

Various embodiments of the present disclosure can be better understood by reference to the following Examples which are offered by way of illustration. The present disclosure is not limited to the Examples given herein.

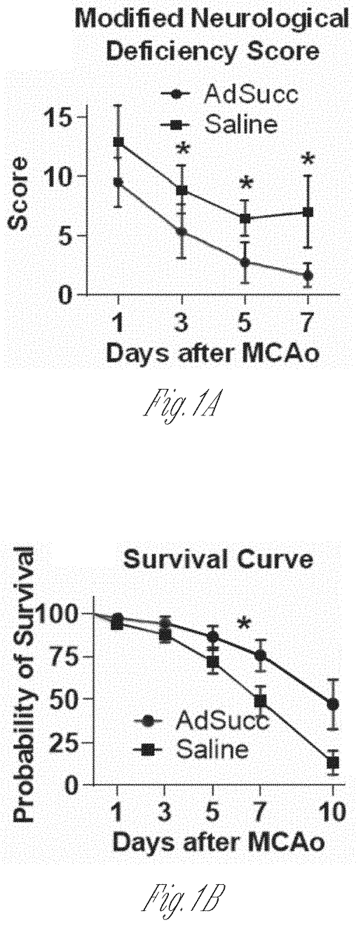

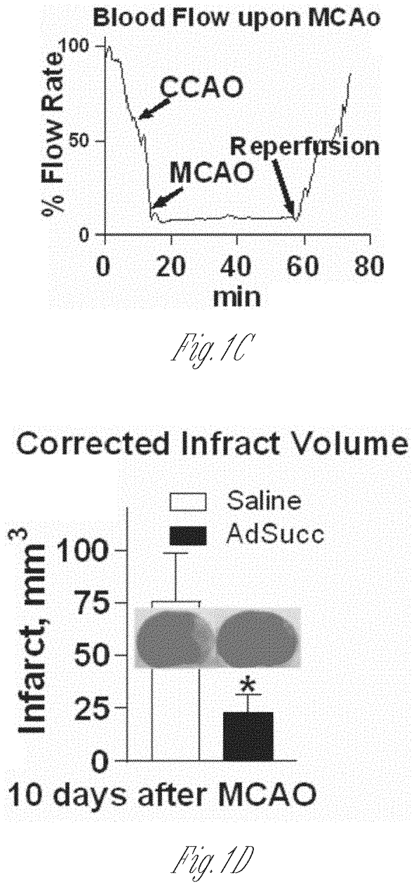

In an example, Stroke was induced in 3 month old mice with 45 min of transient middle cerebral artery occlusion (MCAO) using an intraluminal filament and performed in a double-blind manner. To correct for edema and necrotic tissue loss, corrected infarct volume was calculated as [(volume of the contralateral hemisphere) minus (volume of the viable tissue in ischemic hemisphere)] after triphenyl tetrazolium chloride (TTC) staining on day 7 post-reperfusion. Following MCAO, a modified neurological deficiency score was used to grade neurological function. A series of motor (muscle status, movement, and balance), sensory (visual, tactile, and proprioceptive) and reflex tests were graded (0—being normal and 18—maximal deficit score) by investigator who was blinded to the experimental groups. Mice were tested 1, 3, 5, and 7 days post MCAO surgery. Deaths were plotted and analyzed as survival probability. Mice were treated with AdSucc intranasally 2 h after reperfusion and then every 24 h (10 μL/animal over 10 min, 20 mM in PBS, pH=7.2). This treatment significantly increased brain AdSucc concentrations. This indicates that intranasal AdSucc treatment might be sufficient to induce brain angiogenesis and suggestive that AdSucc has a role as angiogenic factor. These experiments revealed a significant improvement upon AdSucc treatment after stroke injury and suggest a significant potential for AdSucc as a treatment strategy to stimulate angiogenesis for brain injury. Results from this experiment are shown in FIGS. 1A-1D. FIG. 1A is a graph showing neurological deficiency in the test subjects. In FIG. 1A, higher numbers indicate higher deficiency between saline and AdSucc treated animals at the same test day. FIG. 1B is a survival curve of test subjects after MCAO. FIG. 1C is a graph showing representative blood flow rate during MCAO surgery as recorded with a laser Doppler sensor. FIG. 1D is a plot showing the corrected infarct volume seven days after reperfusion calculated as [(contralateral) minus (ipsilateral)viable tissue], n=6, FIG. 1D also shows representative TCC stained brain sections.

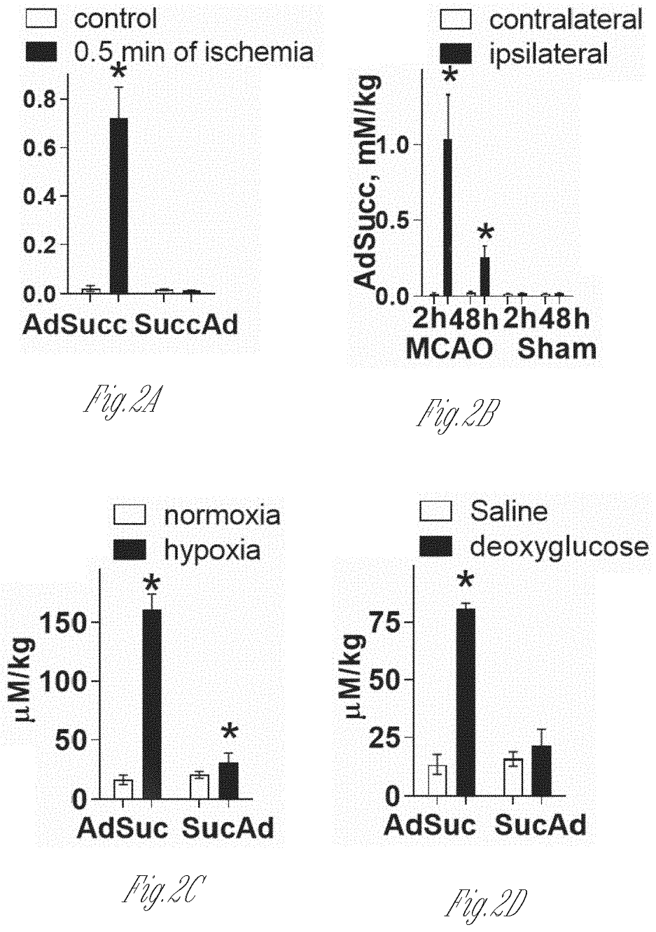

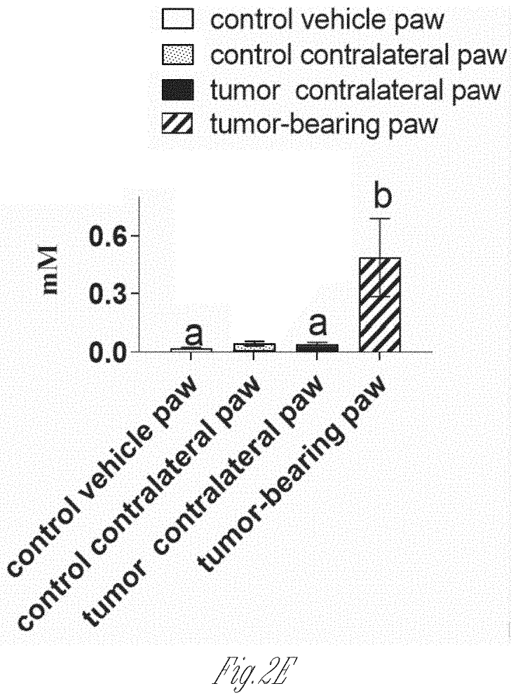

In another example AdSucc was shown to be increased under low energy conditions that require angiogenesis for adaptation. Initially, using an untargeted metabolomics study, an unexpected finding was made of a dramatic, 30-fold increase in AdSucc upon 30 sec of global brain hypoxia/ischemia that reached 0.72±0.12 mM concentration, while SucAd (a product for AdSucc degradation through de-phosphorylation) was unchanged (FIG. 2A). In this example, the metabolome was compared in metabolically quenched brain in situ using microwave irradiation to heat denature brain enzymes to that quenched 30 sec after mouse decapitation, a model for brain global hypoxia/ischemia. Other molecules with similar dynamics upon 30 sec of ischemia have a profound signaling role in the brain including eicosanoids, endocannabinoids, adenosine and other nucleotides. Based on this observation, it is suspected that AdSucc may have a signaling role under low energy conditions. In addition, brain AdSucc was increased in the brain under other low energy conditions: 10-fold upon 2 hours of hypoxia (FIG. 2C), and 6-fold upon glycolysis inhibition with 2-deoxyglucose (FIG. 2D). In the mouse stroke model (2 h), AdSucc was also dramatically increased up to 1.04f0.28 mM/kg in the infarcted, but not in the contralateral hemisphere, these levels remained significantly elevated 48 h post-stroke, but were unaffected in sham operated animals (FIG. 2B). AdSucc was also dramatically induced in a mouse cancer model using implanted carcinoma cells (FIG. 2E) These data further suggest angiogenic AdSucc role under low energy conditions. With reference to FIG. 2, FIGS. 2A-2D relate to brain and carcinoma tumor. FIG. 2E shows AdSucc induction under low energy conditions. In FIG. 2 MCA) is middle cerebral artery occlusion stroke model, analyzed a t 2 hours and 48 hours after reperfusion. Dexoyglucose is an inhibitor for glycolysis under normoxia.

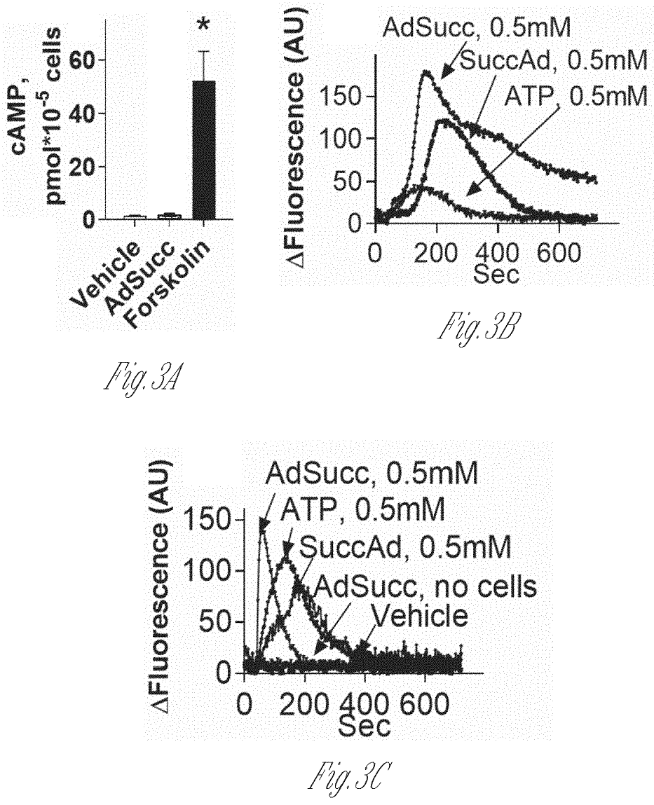

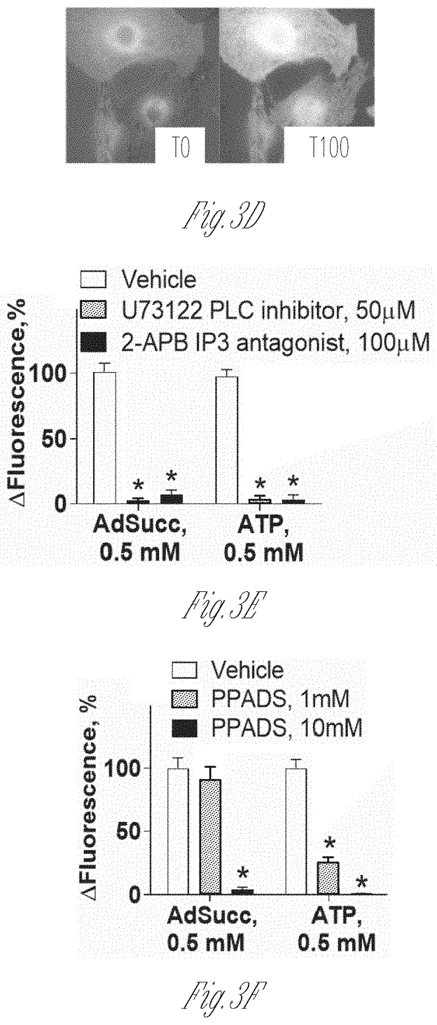

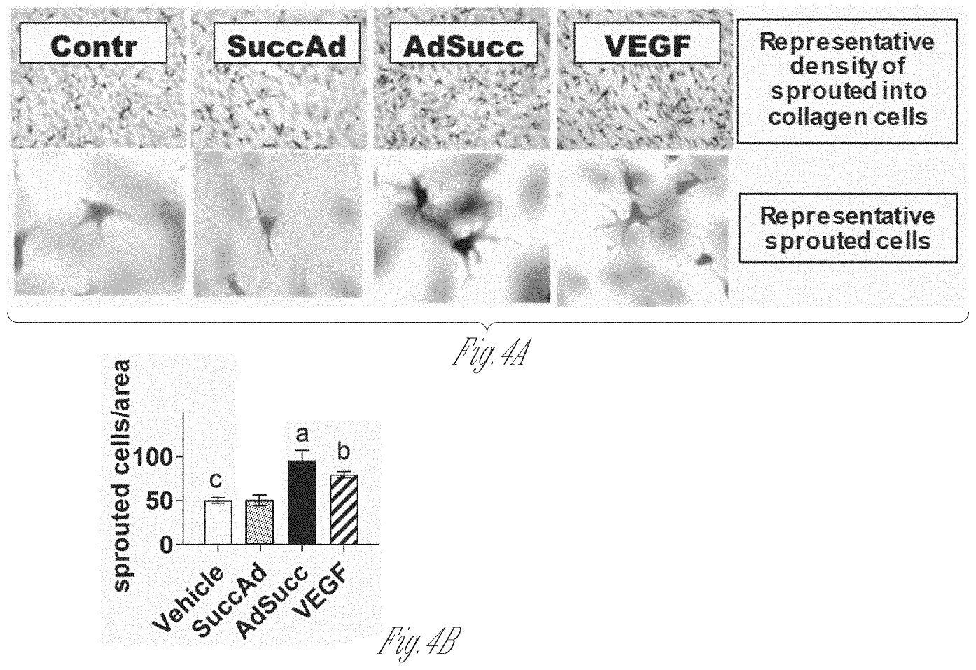

In another example, AdSucc signaling properties were studied. To validate if AdSucc may have a signaling role, the effect of AdSucc on second messenger signaling molecules was studied. AdSucc had no effect on cAMP levels in endothelial cells (FIG. 3A), however, AdSucc, at the concentration found in the ischemic brain, had a very profound effect on intracellular Ca²⁺ levels. Importantly, this Ca²⁺ response was comparable to ATP treatment, and was reproduced in human HUVEC cells and in primary rat microvascular endothelial cells (FIGS. 3B and 3C). Our initial data was collected on a plate reader that averages multicellular responses upon AdSucc stimulation. These findings were independently confirmed with a single cell Ca²⁺ imaging technique (FIG. 3D). To get insights into a mechanism for Ca²⁺ release, cells were treated with phospholipase C (PLC) inhibitors U73122 and inositol phosphate triphosphate (IP3) antagonist 2-APB. Both of these inhibitors significantly attenuated Ca²⁺ response upon AdSucc treatment (FIG. 3E), indicating PLC dependent signal transduction for AdSucc. In addition, transient receptor potential cation channel (TRPC) 4/5 antagonists M084 (100 μM) and ML 204 (25 μM), and TROV4 antagonist HC 067047 (0.5 μM) did not affect AdSucc-induced Ca²⁺ signaling. However, a non-selective P2 purinergic antagonist, PPADS, significantly attenuated the AdSucc-induced Ca²⁺ response in a manner different from the ATP Ca²⁺ response (FIG. 3F), indicating that AdSucc is a possible ligand for P2 purinergic receptors. In FIG. 3, FIG. 3A shows that AdSucc does not change cellular cAMP in HUVEC cells. In FIG. 3B, forskolin was used as a positive control (n=5). In FIG. 3C AdSucc is shown to increase cytosolic Ca²⁺ in the primary rat brain microvascular endothelia 9b) and HUVEC (c) cells as determined using a plate reader, ATP was used as a positive control. FIG. 3D those cellular calcium imaging using 2 micromolar Fura in HUVEC cells at 0 sec and 100 sec (T100) after AdSucc introduction (0.3 mM in PBS) 6E shows PLC-dependent mechanisms for AdSucc effect on HUVEC cellular Ca²⁺ (n=5). 6F shows P2 receptor antagonist PPADS inhibits AdSucc-dependent Ca²⁺ signaling in HUVEC cells (n=5)

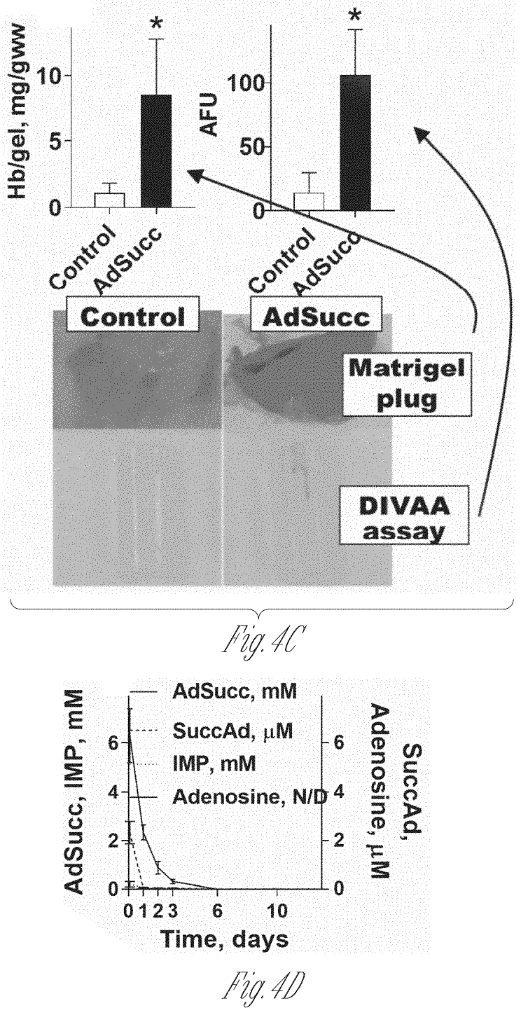

In another example AdSucc's role in angiogenesis was studied. Two different models were used to collect preliminary data in support of AdSucc angiogenic properties. First, an in vitro sprouting assay—a critical step in angiogenesis—was used. HUVEC cells were plated on collagen matrix containing AdSucc, SuccAd, VEGF, or vesicle control for 24 h. For quantification of invaded and sprouted structures, z-stacked images were taking using transmitted light illumination in identical fields following glutaraldehyde fixation and toluidine blue staining. AdSucc significantly potentiated invasion and sprouting of HUVEC cells under normoxia and had a higher effect compared to VEGF (FIG. 4A). The concentrations used (0.5 mM) were in the range found in the brain under low energy conditions. Importantly, SuccAd, a first product in AdSucc degradation, did not have an effect on HUVEC sprouting and this group contained elevated levels of AdSucc degradation products including AMP, IMP, and adenosine indicating that these other products had no impact. These results indicate that AdSucc has stronger sprouting activity compared to its products of hydrolysis, and that AdSucc may have more potent pro-angiogenic properties compared to VEGF under these conditions.

The pro-angiogenic AdSucc effect in vivo using a matrigel plaque loaded with AdSucc (using hemoglobin readout) or DIVA assay kit also loaded with AdSucc (using endothelial cell quantification with FITC labeled lectin) was also studied (FIG. 4B). In both assays, matrigel was implanted into nude mice (s.c.) for 10 days. In both cases, exogenous AdSucc significantly increased angiogenesis in the AdSucc loaded matrigel. In addition, in the matrigel assay, the concentration of AdSucc degradation products, SuccAd, was 1000-fold lower than AdSucc, and adenosine was undetectable in the matrigel (FIG. 4C). Together with the fact that adenine potentiates angiogenesis through stabilizing HIF1/2, and AdSucc is induced upon glycolysis inhibition under normoxia (FIG. 4D), this preliminary data indicated that AdSucc is a novel pro-angiogenic factor which expresses pro-angiogenic activity independently from its possible products of degradation, and can act independently from O₂ availability. In FIGS. 4A-4D, FIG. 4A and FIG. 4B show that AdSucc increases HUVEC cells invasion and sprouting under concentration found in ischemic brain (0.5 mM, n=4). FIG. 4C shows AdSucc (0.5 mM) potentiates angiogenesis in the matrigel (quantified by hemoglobin) and DIVA (quantified with lectin FITS to label endothelial cells) in vivo assays, n=4. FIG. 4D shows matrigel analysis for products of AdSucc degradation upon incubation in vivo (n=3).

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Embodiments

The following exemplary embodiments are provided, the numbering of which is not to be construed as designating levels of importance:

Example 1 provides a pharmaceutical composition, the composition comprising the structure according to Formula I or pharmaceutically acceptable ester thereof, prodrug thereof, or a pharmaceutically acceptable salt thereof:

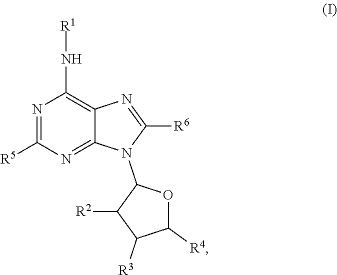

wherein R¹, R², R³, R⁴, R⁵, and R⁶, are independently selected from the group consisting of —H, —OH, —COOH, and substituted or unsubstituted (C₁-C₂₀)hydrocarbyl.

Example 2 provides the pharmaceutical composition of Example 1, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 3 provides the pharmaceutical composition of any one of Examples 1 or 2, comprising the structure according to any one of Formulas H, III, or a mixture thereof or pharmaceutically acceptable ester thereof, prodrug thereof, or a pharmaceutically acceptable salt thereof:

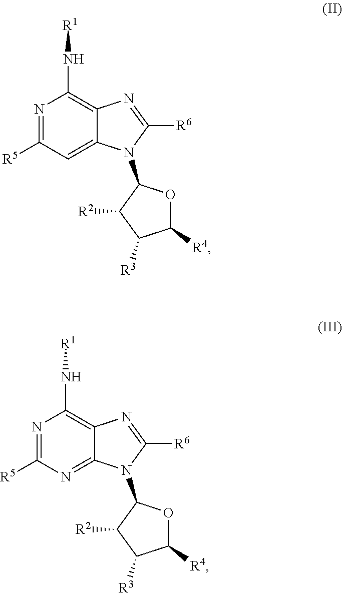

wherein R¹, R², R³, R⁴, R⁵, and R⁶, are independently selected from the group consisting of —H, —OH, and substituted or unsubstituted (C₁-C₂₀)hydrocarbyl.

Example 4 provides the pharmaceutical composition of Example 3, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 5 provides the pharmaceutical composition of any one of Examples 1-4, comprising the structure according to any one of Formulas IV, V, or a mixture thereof or pharmaceutically acceptable ester thereof, prodrug thereof, or a pharmaceutically acceptable salt thereof:

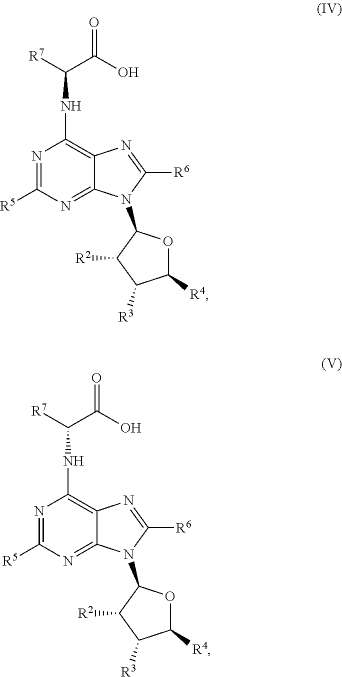

wherein R², R³, R⁴, R⁵, R⁶, and R⁷, are independently selected from the group consisting of —H, —OH, —COOH, and substituted or unsubstituted (C₁-C₂₀)hydrocarbyl.

Example 6 provides the pharmaceutical composition of Example 5, wherein R², R³, R⁴, R⁵, R⁶, and R⁷, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 7 provides the pharmaceutical composition of any one of Examples 1-6, comprising the structure according to any one of Formulas VI, VII, or a mixture thereof or pharmaceutically acceptable ester thereof, prodrug thereof, or a pharmaceutically acceptable salt thereof:

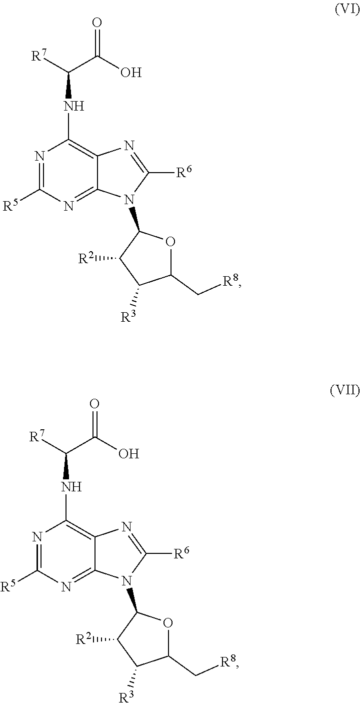

wherein R², R³, R⁵, R⁶, R⁷, and R⁸, are independently selected from the group consisting of —H, —OH, —COOH, and substituted or unsubstituted (C₁-C₂₀)hydrocarbyl.

Example 8 provides the pharmaceutical composition of Example 7, wherein R², R³, R⁵, R⁶, R⁷, and R⁸, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 9 provides the pharmaceutical composition of any one of Examples 1-8, comprising the structure according to any one of Formulas VIII, IX, X, XI, or a mixture thereof, pharmaceutically acceptable ester thereof, prodrug thereof, or a pharmaceutically acceptable salt thereof:

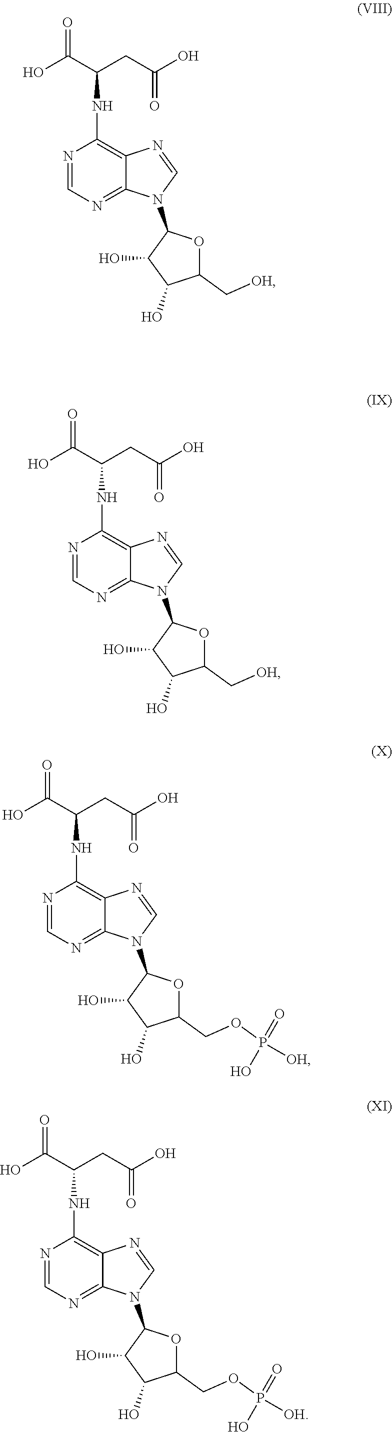 [SMILES: O=C(O)C[C@@H](Nc1ncnc2c1ncn2[C@H]1OC(CO)[C@H](O)[C@@H]1O)C(=O)O ; O=C(O)C[C@H](Nc1ncnc2c1ncn2[C@H]1OC(CO)[C@H](O)[C@@H]1O)C(=O)O ; O=C(O)C[C@@H](Nc1ncnc2c1ncn2[C@H]1OC(COP(=O)(O)O)[C@H](O)[C@@H]1O)C(=O)O ; O=C(O)C[C@H](Nc1ncnc2c1ncn2[C@H]1OC(COP(=O)(O)O)[C@H](O)[C@@H]1O)C(=O)O]

Example 10 provides a pharmaceutical composition for promoting angiogenesis, the composition comprising the structure according to Formula I or pharmaceutically acceptable ester thereof, prodrug thereof, or a pharmaceutically acceptable salt thereof:

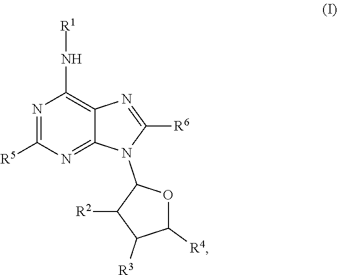

wherein R¹, R², R³, R⁴, R⁵, and R⁶, are independently selected from the group consisting of —H, OH, —COOH, and substituted or unsubstituted (C₁-C₂₀)hydrocarbyl.

Example 11 provides the pharmaceutical composition for promoting angiogenesis of Example 10, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 12 provides the pharmaceutical composition for promoting angiogenesis of any one of Examples 10 or 11, comprising the structure according to any one of Formulas II, III, or a mixture thereof or pharmaceutically acceptable ester thereof, prodrug thereof, or a pharmaceutically acceptable salt thereof:

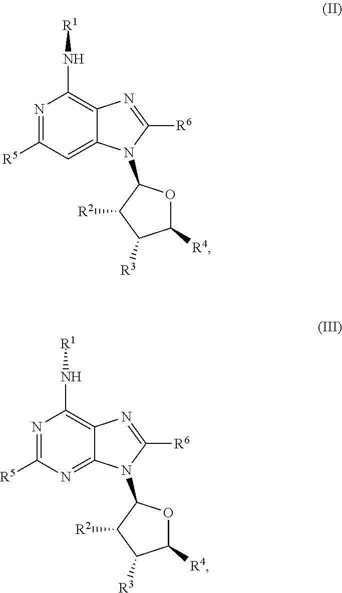

wherein R¹, R², R³, R⁴, R⁵, and R⁶, are independently selected from the group consisting of —H, —OH, —COOH, and substituted or unsubstituted (C₁-C₂₀)hydrocarbyl.

Example 13 provides the pharmaceutical composition for promoting angiogenesis of Example 12, wherein R¹, R², R³, R⁴, R⁵, and R⁶, are independently selected from the group consisting of —H, —OH, —COOH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 14 provides the pharmaceutical composition for promoting angiogenesis of any one of Examples 10-13, comprising the structure according to any one of Formulas IV, V, or a mixture thereof or pharmaceutically acceptable ester thereof, prodrug thereof, or a pharmaceutically acceptable salt thereof:

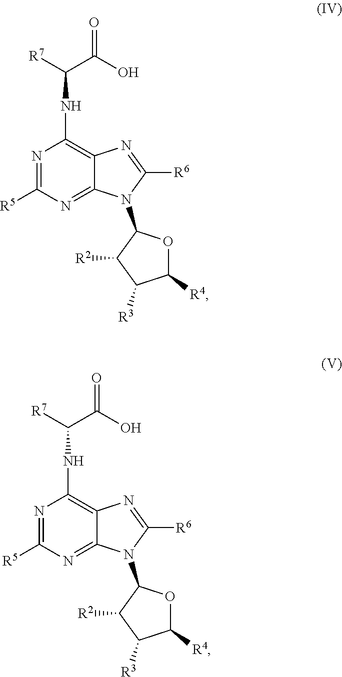

wherein R², R³, R⁴, R⁵, R⁶, and R⁷, are independently selected from the group consisting of —H, —OH, —COOH, and substituted or unsubstituted (C₁-C₂₀)hydrocarbyl.

Example 15 provides the pharmaceutical composition for promoting angiogenesis of Example 14, wherein R², R³, R⁴, R⁵, R⁶, and R⁷, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 16 provides the pharmaceutical composition for promoting angiogenesis of any one of Examples 10-15, comprising the structure according to any one of Formulas VI, VII, or a mixture thereof or pharmaceutically acceptable ester thereof, prodrug thereof, or a pharmaceutically acceptable salt thereof:

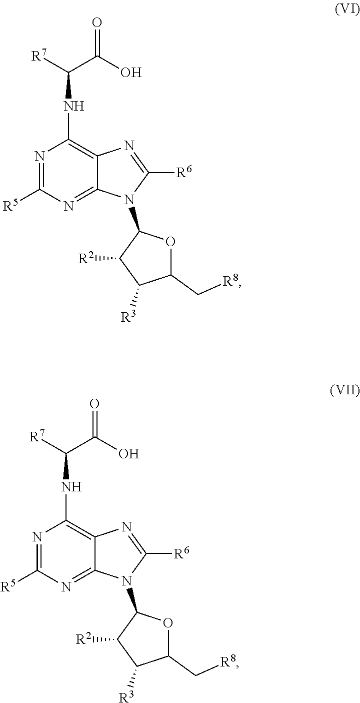

wherein R², R³, R⁵, R⁶, R⁷, and R⁸, are independently selected from the group consisting of —H, —OH, —COOH, and substituted or unsubstituted (C₁-C₂₀)hydrocarbyl.

Example 17 provides the pharmaceutical composition for promoting angiogenesis of Example 16, wherein R², R³, R⁵, R⁶, R⁷, and R⁸, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 18 provides the pharmaceutical composition for promoting angiogenesis of any one of Examples 10-17, comprising the structure according to any one of Formulas VIII, IX, X, XI, or a mixture thereof, pharmaceutically acceptable ester thereof, prodrug thereof, or a pharmaceutically acceptable salt thereof:

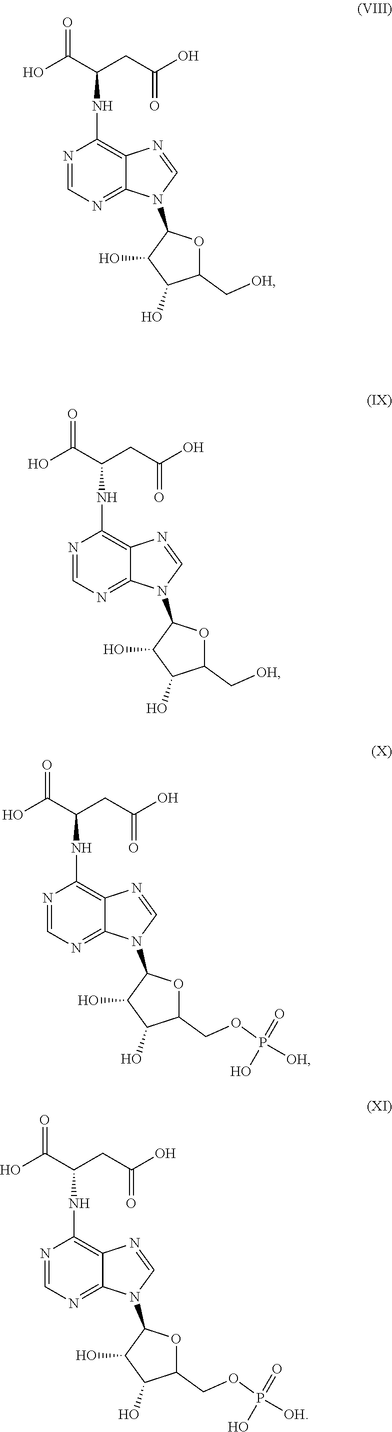 [SMILES: O=C(O)C[C@@H](Nc1ncnc2c1ncn2[C@H]1OC(CO)[C@H](O)[C@@H]1O)C(=O)O ; O=C(O)C[C@H](Nc1ncnc2c1ncn2[C@H]1OC(CO)[C@H](O)[C@@H]1O)C(=O)O ; O=C(O)C[C@@H](Nc1ncnc2c1ncn2[C@H]1OC(COP(=O)(O)O)[C@H](O)[C@@H]1O)C(=O)O ; O=C(O)C[C@H](Nc1ncnc2c1ncn2[C@H]1OC(COP(=O)(O)O)[C@H](O)[C@@H]1O)C(=O)O]

Example 19 provides a method for promoting angiogenesis in a subject, the method administering a composition comprising the structure according to Formula I or pharmaceutically acceptable ester thereof, prodrug thereof, or a pharmaceutically acceptable salt thereof:

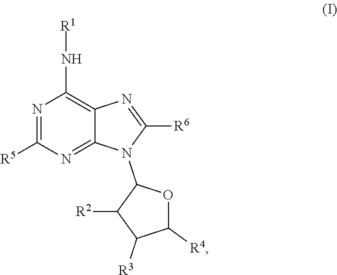

wherein R¹, R², R³, R⁴, R⁵, and R⁶, are independently selected from the group consisting of —H, —OH, —COOH, and substituted or unsubstituted (C₁-C₂₀)hydrocarbyl.

Example 20 provides the method for promoting angiogenesis of Example 19, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 21 provides the method for promoting angiogenesis of any one of Examples 19 or 20, comprising the structure according to any one of Formulas II, III, or a mixture thereof or pharmaceutically acceptable ester thereof, prodrug thereof, or a pharmaceutically acceptable salt thereof:

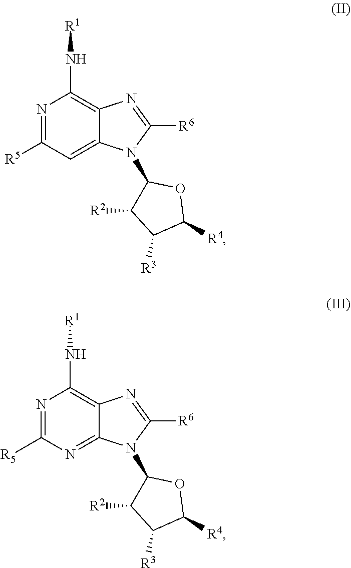

wherein R¹, R², R³, R⁴, R⁵, and R⁶, are independently selected from the group consisting of —H, —OH, —COOH, and substituted or unsubstituted (C₁-C₂₀)hydrocarbyl.

Example 22 provides the method for promoting angiogenesis of Example 19, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂M)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 23 provides the method for promoting angiogenesis of any one of Examples 19-22, comprising the structure according to any one of Formulas IV, V, or a mixture thereof or pharmaceutically acceptable ester thereof, prodrug thereof, or a pharmaceutically acceptable salt thereof:

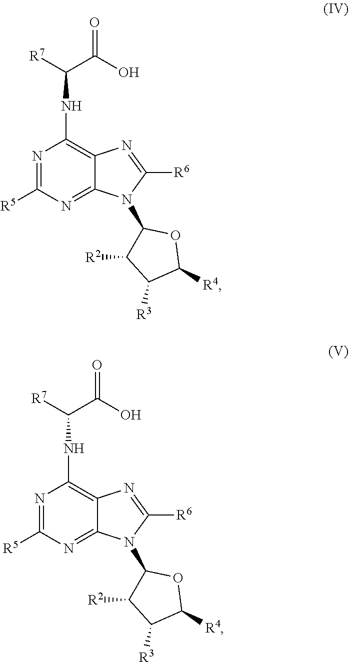

wherein R², R³, R⁴, R⁵, R⁶, and R⁷, are independently selected from the group consisting of —H, —OH, —COOH, and substituted or unsubstituted (C₁-C₂₀)hydrocarbyl.

Example 24 provides the method for promoting angiogenesis of Example 23, wherein R², R³, R⁴, R⁵, R⁶, and R⁷, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 25 provides the method for promoting angiogenesis of any one of Examples 19-24, comprising the structure according to any one of Formulas VI, VII, or a mixture thereof or pharmaceutically acceptable ester thereof, prodrug thereof, or a pharmaceutically acceptable salt thereof:

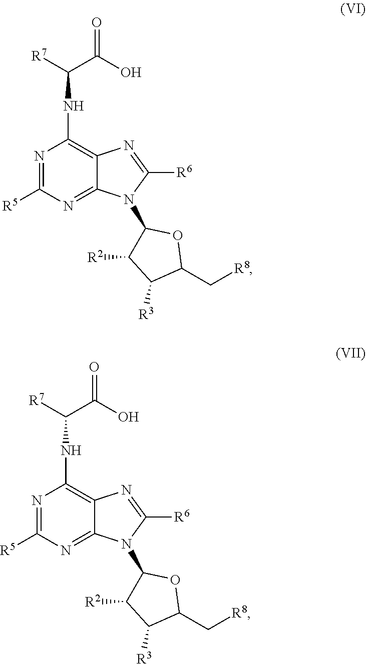

wherein R², R³, R⁵, R⁶, R⁷, and R⁸, are independently selected from the group consisting of —H, —OH, —COOH, and substituted or unsubstituted (C₁-C₂₀)hydrocarbyl.

Example 26 provides the method for promoting angiogenesis of Example 25, wherein R², R³, R⁵, R⁶, R⁷, and R⁸, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

Example 27 provides the method for promoting angiogenesis of any one of Examples 19-26, comprising the structure according to any one of Formulas VIII, IX, X, XI, or a mixture thereof, pharmaceutically acceptable ester thereof, prodrug thereof, or a pharmaceutically acceptable salt thereof:

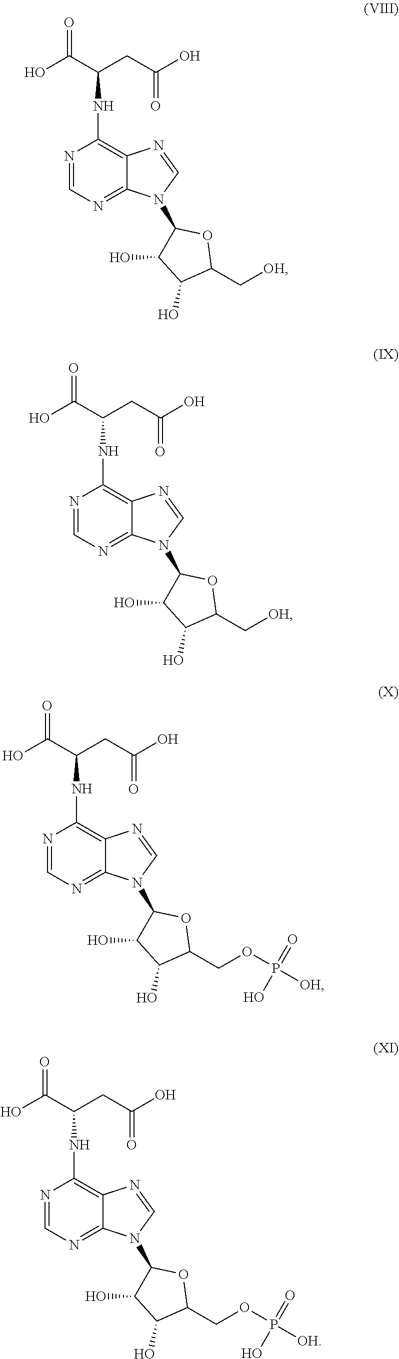 [SMILES: O=C(O)C[C@@H](Nc1ncnc2c1ncn2[C@H]1OC(CO)[C@H](O)[C@@H]1O)C(=O)O ; O=C(O)C[C@H](Nc1ncnc2c1ncn2[C@H]1OC(CO)[C@H](O)[C@@H]1O)C(=O)O ; O=C(O)C[C@@H](Nc1ncnc2c1ncn2[C@H]1OC(COP(=O)(O)O)[C@H](O)[C@@H]1O)C(=O)O ; O=C(O)C[C@H](Nc1ncnc2c1ncn2[C@H]1OC(COP(=O)(O)O)[C@H](O)[C@@H]1O)C(=O)O]

1. A pharmaceutical composition, the composition comprising the structure according to Formula I or pharmaceutically acceptable ester thereof, prodrug thereof, or a pharmaceutically acceptable salt thereof: 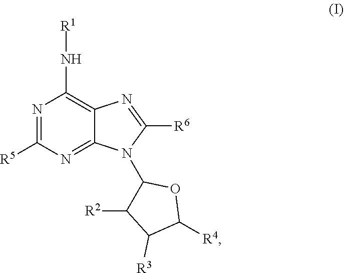
wherein R¹, R², R³, R⁴, R⁵, and R⁶, are independently selected from the group consisting of —H, —OH, —COOH, and substituted or unsubstituted (C₁-C₂₀)hydrocarbyl.

2. The pharmaceutical composition of claim 1, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

3. The pharmaceutical composition of claim 1, comprising the structure according to any one of Formulas II, III, or a mixture thereof or pharmaceutically acceptable ester thereof, prodrug thereof, or a pharmaceutically acceptable salt thereof: 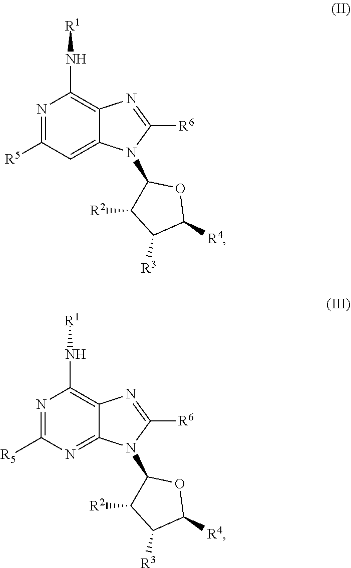
wherein R¹, R², R³, R⁴, R⁵, and R⁶, are independently selected from the group consisting of —H, —OH, —COOH, and substituted or unsubstituted (C₁-C₂₀)hydrocarbyl.

4. The pharmaceutical composition of claim 3, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

5. The pharmaceutical composition of claim 1, comprising the structure according to any one of Formulas IV, V, or a mixture thereof or pharmaceutically acceptable ester thereof, prodrug thereof, or a pharmaceutically acceptable salt thereof: 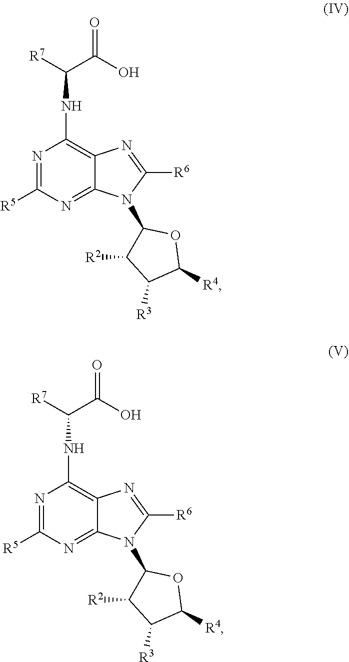
wherein R², R³, R⁴, R⁵, R⁶, and R⁷, are independently selected from the group consisting of —H, —OH, —COOH, and substituted or unsubstituted (C₁-C₂₀)hydrocarbyl.

6. The pharmaceutical composition of claim 5, wherein R², R³, R⁴, R⁵, R⁶, and R⁷, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

7. The pharmaceutical composition of claim 1, comprising the structure according to any one of Formulas VI, VII, or a mixture thereof or pharmaceutically acceptable ester thereof, prodrug thereof, or a pharmaceutically acceptable salt thereof: 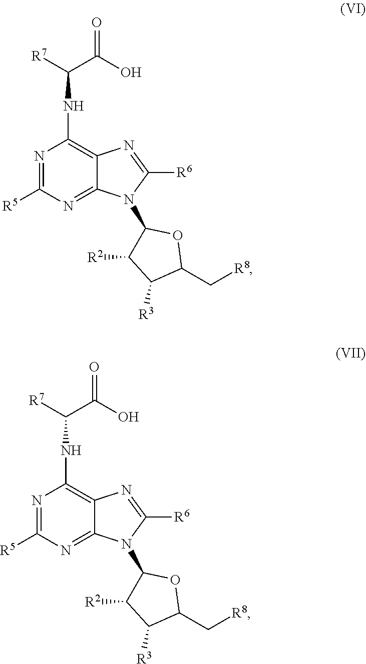
wherein R², R³, R⁵, R⁶, R⁷, and R⁸, are independently selected from the group consisting of —H, —OH, —COOH, and substituted or unsubstituted (C₁-C₂₀)hydrocarbyl.

8. The pharmaceutical composition of claim 7, wherein R², R³, R⁵, R⁶, R⁷, and R⁸, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

9. The pharmaceutical composition of claim 1, comprising the structure according to any one of Formulas VIII, IX, X, XI, or a mixture thereof, pharmaceutically acceptable ester thereof, prodrug thereof, or a pharmaceutically acceptable salt thereof:

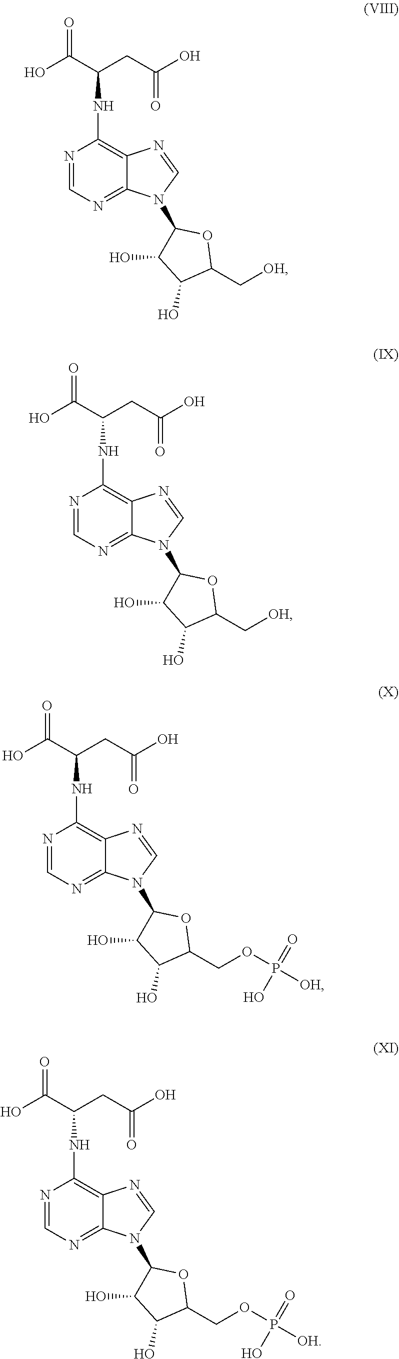 [SMILES: O=C(O)C[C@@H](Nc1ncnc2c1ncn2[C@H]1OC(CO)[C@H](O)[C@@H]1O)C(=O)O ; O=C(O)C[C@H](Nc1ncnc2c1ncn2[C@H]1OC(CO)[C@H](O)[C@@H]1O)C(=O)O ; O=C(O)C[C@@H](Nc1ncnc2c1ncn2[C@H]1OC(COP(=O)(O)O)[C@H](O)[C@@H]1O)C(=O)O ; O=C(O)C[C@H](Nc1ncnc2c1ncn2[C@H]1OC(COP(=O)(O)O)[C@H](O)[C@@H]1O)C(=O)O]

10. A pharmaceutical composition for promoting angiogenesis, the composition comprising the structure according to Formula I or pharmaceutically acceptable ester thereof, prodrug thereof, or a pharmaceutically acceptable salt thereof: 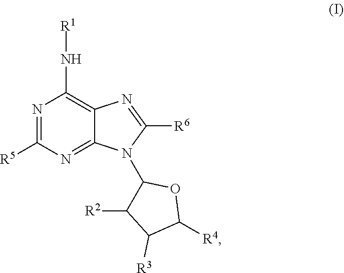
wherein R¹, R², R³, R⁴, R⁵, and R⁶, are independently selected from the group consisting of —H, —OH, —COOH, and substituted or unsubstituted (C₁-C₂₀)hydrocarbyl.

11. The pharmaceutical composition for promoting angiogenesis of claim 10, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

12. The pharmaceutical composition for promoting angiogenesis of claim 10, comprising the structure according to any one of Formulas II, III, or a mixture thereof or pharmaceutically acceptable ester thereof, prodrug thereof, or a pharmaceutically acceptable salt thereof: 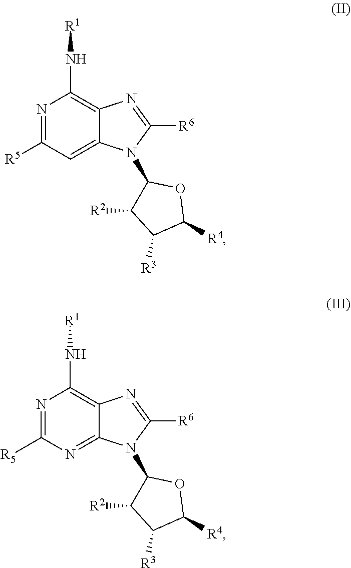
wherein R¹, R², R³, R⁴, R⁵, and R⁶, are independently selected from the group consisting of —H, —OH, —COOH, and substituted or unsubstituted (C₁-C₂₀)hydrocarbyl.

13. The pharmaceutical composition for promoting angiogenesis of claim 12, wherein R¹, R², R³, R⁴, R⁵, and R⁵, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

14. The pharmaceutical composition for promoting angiogenesis of claim 10, comprising the structure according to any one of Formulas IV, V, or a mixture thereof or pharmaceutically acceptable ester thereof, prodrug thereof, or a pharmaceutically acceptable salt thereof: 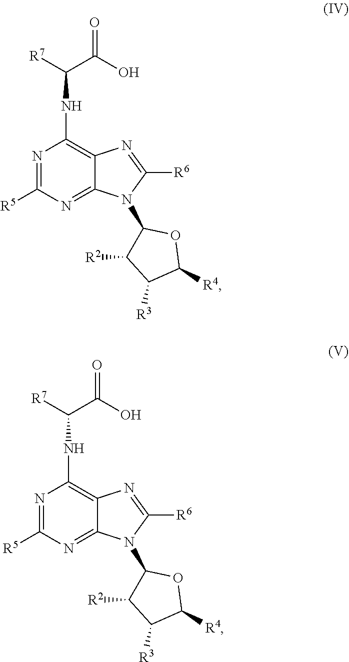
wherein R², R³, R⁴, R⁵, R⁶, and R⁷, are independently selected from the group consisting of —H, —OH, —COOH, and substituted or unsubstituted (C₁-C₂₀)hydrocarbyl.

15. The pharmaceutical composition for promoting angiogenesis of claim 14, wherein R², R³, R⁴, R⁵, R⁶, and R⁷, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

16. The pharmaceutical composition for promoting angiogenesis of claim 10, comprising the structure according to any one of Formulas VI, VII, or a mixture thereof or pharmaceutically acceptable ester thereof, prodrug thereof, or a pharmaceutically acceptable salt thereof: 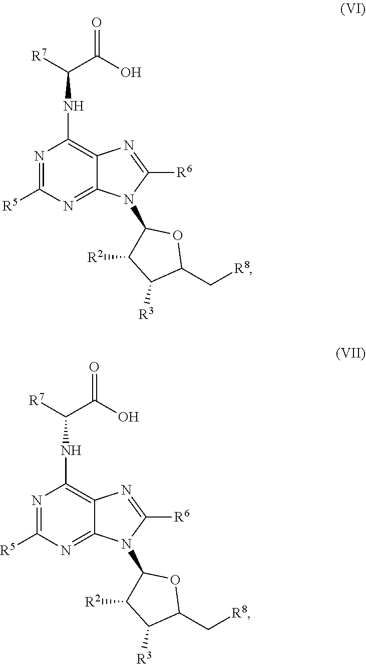
wherein R², R³, R⁵, R⁶, R⁷, and R⁸, are independently selected from the group consisting of —H, —OH, —COOH, and substituted or unsubstituted (C₁-C₂₀)hydrocarbyl.

17. The pharmaceutical composition for promoting angiogenesis of claim 16, wherein R², R³, R⁵, R⁶, R⁷, and R⁸, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.

18. The pharmaceutical composition for promoting angiogenesis of claim 10, comprising the structure according to any one of Formulas VIII, IX, X, XI, or a mixture thereof, pharmaceutically acceptable ester thereof, prodrug thereof, or a pharmaceutically acceptable salt thereof:

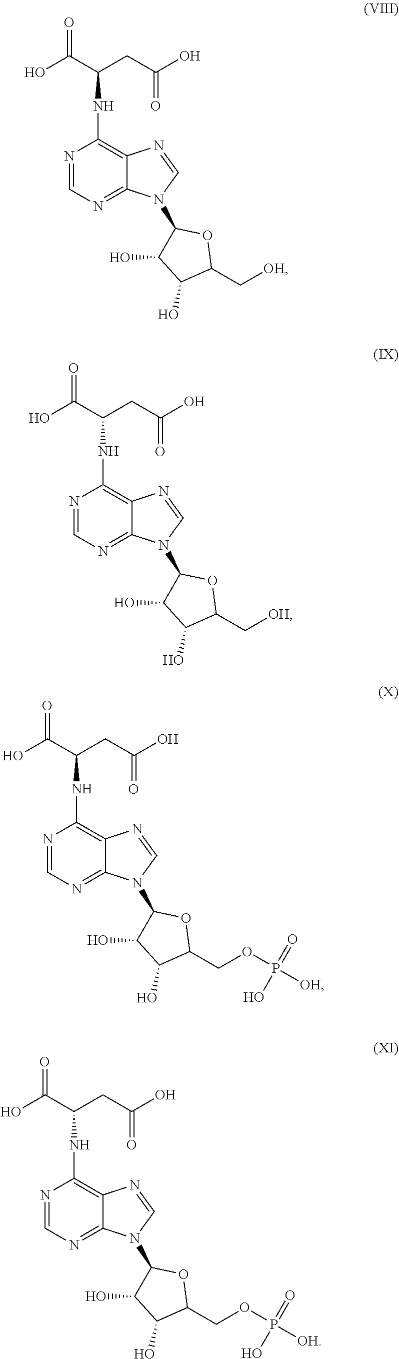 [SMILES: O=C(O)C[C@@H](Nc1ncnc2c1ncn2[C@H]1OC(CO)[C@H](O)[C@@H]1O)C(=O)O ; O=C(O)C[C@H](Nc1ncnc2c1ncn2[C@H]1OC(CO)[C@H](O)[C@@H]1O)C(=O)O ; O=C(O)C[C@@H](Nc1ncnc2c1ncn2[C@H]1OC(COP(=O)(O)O)[C@H](O)[C@@H]1O)C(=O)O ; O=C(O)C[C@H](Nc1ncnc2c1ncn2[C@H]1OC(COP(=O)(O)O)[C@H](O)[C@@H]1O)C(=O)O]

19. A method for promoting angiogenesis in a subject, the method administering a composition comprising the structure according to Formula I or pharmaceutically acceptable ester thereof, prodrug thereof, or a pharmaceutically acceptable salt thereof: 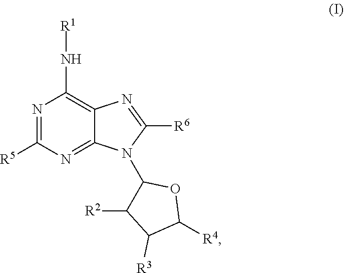
wherein R¹, R², R³, R⁴, R⁵, and R⁶, are independently selected from the group consisting of —H, —OH, —COOH, and substituted or unsubstituted (C₁-C₂₀)hydrocarbyl, wherein angiogenesis is generated to treat stroke, neurodegeneration, metabolic brain disorders, muscular dystrophy, or a combination thereof.

20. The method for promoting angiogenesis of claim 19, wherein R¹, R², R³, R⁴, R⁵, and R⁶, are independently selected from the group consisting of —H, —OH, substituted or unsubstituted (C₁-C₂₀)alkyl, (C₂-C₂₀)alkenyl, (C₂-C₂₀)alkynyl, (C₁-C₂₀)acyl, (C₁-C₂₀)alkoxy, an amine, (C₁-C₂₀)haloalkyl, (C₃-C₂₀)cycloalkyl, (C₃-C₂₀)heterocycloalkyl, (C₃-C₂₀)aryl, (C₃-C₂₀)aralkyl, (C₃-C₂₀)heteroaralkyl.
